(19) United States
(12) Patent Application Publication
(10) Pub. No.: US 2009/0118811 A1
(43) Pub. Date: May 7, 2009

(54) Globe Stent

(75) Inventor: Noreen Moloney, Moycullen (IE)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 11/934,854

(22) Filed: Nov. 5, 2007

(51) Int. Cl. A61F 2/84; A61F 2/82

(52) U.S. Cl. 623/1.12; 623/1.15; 623/1.18; 623/1.2

(57) ABSTRACT

A stent for treating a region of a body lumen wherein at least two vessels form a junction includes a compressed state and an expanded state. In the expanded state, the stent is generally an ellipsoidal, spheroidal, or spherical shape. The stent is delivered to the junction in the compressed state disposed within a sleeve. Once at the junction, the sleeve is withdrawn proximally relative to the stent such that the stent is released from the sleeve and expands to the expanded state. A balloon may further expand the stent to appose the walls of the body lumen at the junction.

### FIELD OF THE INVENTION

The invention relates generally stents and grafts for supporting strictures or stenoses in the human body. More particularly, the invention relates to a stent or graft for treating site or sites at or near a bifurcation or trifurcation of a body lumen.

### BACKGROUND OF THE INVENTION

Stents are generally cylindrical-shaped devices that are radially expandable to hold open a segment of a vessel or other anatomical lumen after implantation into the lumen. Various types of stents are in use, including expandable and self-expanding stents. Expandable stents generally are conveyed to the area to be treated on balloon catheters or other expandable devices. For insertion, the stent is positioned in a compressed configuration along the delivery device, for example crimped onto a balloon that is folded or otherwise wrapped about a guide wire that is part of the delivery device. After the stent is positioned across the lesion, it is expanded by the delivery device, causing the diameter of the stent to expand. For a self-expanding stent, commonly a sheath is retracted, allowing expansion of the stent.

Stents are used in conjunction with balloon catheters in a variety of medical therapeutic applications, including intravascular angioplasty. For example, a balloon catheter device is inflated during percutaneous transluminal coronary angioplasty (PTCA) to dilate a stenotic blood vessel. The stenosis may be the result of a lesion such as a plaque or thrombus. When inflated, the pressurized balloon exerts a compressive force on the lesion, thereby increasing the inner diameter of the affected vessel. The increased interior vessel diameter facilitates improved blood flow.

Soon after the procedure, however, a significant proportion of treated vessels restenose. To prevent restenosis, a stent, constructed of a metal or polymer, is implanted within the vessel to maintain lumen size. The stent acts as a scaffold to support the lumen in an open position. Configurations of stents include a cylindrical tube defined by a solid wall, a mesh, interconnected stents, or like segments. Exemplary stents are disclosed in U.S. Pat. No. 5,292,331 to Boneau, U.S. Pat. No. 6,090,127 to Globerman, U.S. Pat. No. 5,133,732 to Wiktor, U.S. Pat. No. 4,739,762 to Palmaz, and U.S. Pat. No. 5,421,955 to Lau.

Difficulties arise when the area requiring treatment is located near a bifurcation, the point at which a single vessel branches into two vessels, or other junction where several vessels meet or branch off (such as a trifurcation). To effectively treat a vascular condition at a bifurcation or trifurcation, the stent must cover the entire affected area without obstructing blood flow in the adjoining vessels. This can be quite difficult to achieve.

Various conventional stenting techniques have been disclosed for treating bifurcations. One conventional bifurcation stenting technique includes first stenting the side-branch vessel and then the main vessel. Angle variations or limited visualization at the ostium (area at the opening) of the side-branch vessel may prevent accurate placement of the side-branch stent, resulting in the stent providing suboptimal coverage of the ostium or in the stent protruding into the main vessel and interfering with blood flow. The stent may, additionally, block access to portions of the adjoining vessel that require further intervention.

Another conventional technique involves first stenting the main vessel and then advancing a second stent through the wall of the main vessel stent and into the side-branch vessel, where the second stent is deployed. Disadvantages of this method include a risk of compressing the ostium of the side branch vessel when the main vessel stent is deployed, making insertion of a second stent difficult, if not impossible. Even when the side-branch vessel remains open, accurate positioning of a second stent through the wall of the first stent and into the side branch presents significant challenges and may result in undesirable overlapping of the stents.

Where the bifurcation forms a Y-shape, with the main vessel branching into two smaller vessels, conventional techniques have included placing three stents, one within the main vessel, and one within each of the smaller vessels. The problems discussed above may be present with this technique, as well.

Devices developed specifically to address the problems that arise in the treatment of stenoses at or near the site of a bifurcation of a body lumen are known in the art. Examples of catheters for use in treating bifurcated lumens or delivery systems for bifurcated endoluminal prostheses are shown in U.S. Pat. No. 5,720,735 to Dorros, U.S. Pat. No. 5,669,924 to Shaknovich, U.S. Pat. No. 5,749,825 to Fischell, et al., and U.S. Pat. No. 5,718,724 to Goicoechea et al.

Various techniques have been used to deliver multiple prostheses in order to provide radial support to both a main blood vessel, for example, and contemporaneously to side branches of the blood vessel. Further, single bifurcated stents and grafts have been developed in order to treat such conditions at the site of a branch of a body lumen. A bifurcated stent and/or graft typically comprises a tubular body or trunk and two tubular legs. Examples of bifurcated stents are shown in U.S. Pat. No. 5,723,004 to Dereume et al., U.S. Pat. No. 4,994,071 to MacGregor, and European Pat. Application EP 0 804 907 A2 to Richter, et al.

Conventional bifurcated stents tend to focus on the branched vessels themselves, rather than the junction where the vessel meet or branch off from. The junction may be shaped such that conventional bifurcated stents or individual stents placed in each of the branch vessels do not adequately support the junction. Hence, there is a need for a stent that adequately supports the junction of a bifurcated, trifurcated, or other branched vessel.

### BRIEF SUMMARY OF THE INVENTION

The present invention is directed to a stent for treating a region where at least two vessels form a junction. The stent in its compressed state is small enough to be delivered intravascularly through the vessel to the junction. In its expanded state, the stent is generally ellipsoidal, spheroidal, or spherically shaped such that it supports the vessel at the junction. The stent is preferably a self-expanding stent made from a shape memory material.

The present invention is further directed to a method for treating a region of a body lumen wherein at least two vessels form a junction. The stent is disposed within a sleeve in its compressed state. The sleeve and stent are then delivered to the junction. The sleeve is then withdrawn proximally relative to the stent such that the stent is released from the sleeve, wherein the stent expands to form a generally ellipsoidal, spheroidal, or spherical shape. A balloon catheter may be advanced to the junction prior to the stent to perform a balloon angioplasty at the junction site. Further, the stent may be mounted on a balloon catheter to further expand the stent to appose the vessel walls at the junction after the stent is released from the sleeve.

### BRIEF DESCRIPTION OF DRAWINGS

The foregoing and other features and advantages of the invention will be apparent from the following description of the invention as illustrated in the accompanying drawings. The accompanying drawings, which are incorporated herein and form a part of the specification, further serve to explain the principles of the invention and to enable a person skilled in the pertinent art to make and use the invention. The drawings are not to scale.

FIG. 1 illustrates a junction of four vessel branches with lesions disposed at the junction.

FIG. 2 illustrates an ellipsoidal stent in accordance with an embodiment of the present invention.

FIG. 3 illustrates a spherical stent in accordance with another embodiment of the present invention.

FIG. 4 illustrates the stent of FIG. 2 disposed in a sleeve and being delivered to the junction of FIG. 1.

FIG. 5 illustrates the delivery system of FIG. 4 as the sleeve approaches the junction.

FIG. 6 illustrates the delivery system of FIG. 4 as the sleeve is moved proximally relative to the stent to release the stent from the sleeve.

FIG. 7 illustrates the stent of FIG. 2 deployed at the junction of FIG. 1.

FIG. 8 illustrates a balloon catheter being delivered to a junction of a bifurcated vessel.

FIG. 9 illustrates the balloon catheter of FIG. 8 with the balloon expanded at the junction.

FIG. 10 illustrates the stent of FIG. 3 being delivered to the junction of a bifurcated vessel.

FIG. 11 illustrates the delivery system of FIG. 10 as the sleeve is moved proximally relative to the stent to release the stent from the sleeve.

FIG. 12 illustrates the stent of FIG. 3 deployed at the junction of FIG. 8.

FIG. 13 illustrates a stent mounted on a balloon catheter and disposed within a sleeve in accordance with an embodiment for delivering a stent in accordance with the present invention.

### DETAILED DESCRIPTION OF THE INVENTION

Specific embodiments of the present disclosure are now described with reference to the figures, wherein like reference numbers indicate identical or functionally similar elements. The terms “distal” and “proximal” are used in the following description with respect to a position or direction relative to the treating clinician. “Distal” or “distally” are a position distant from or in a direction away from the clinician. “Proximal” and “proximally” are a position near or in a direction toward the clinician.

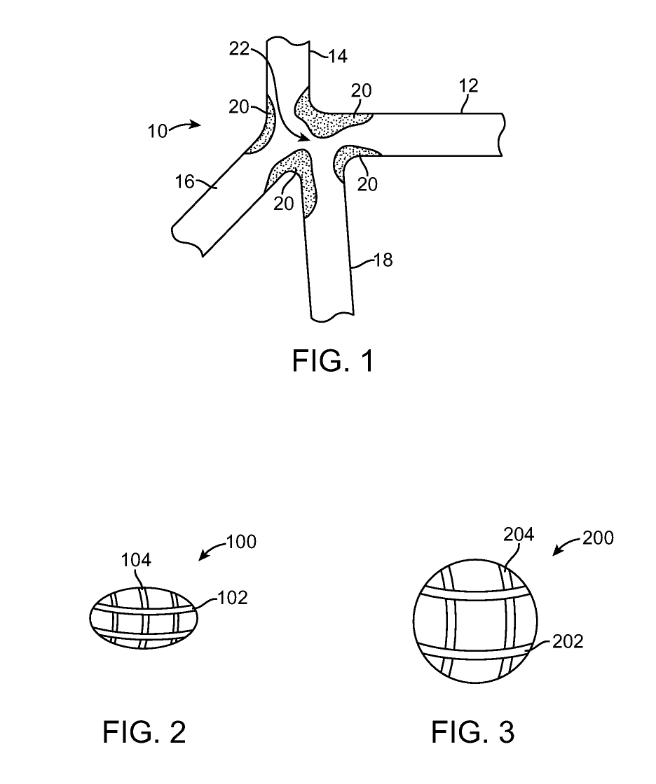

FIG. 1 shows a trifurcated vessel 10 including a first vessel branch 12, a second vessel branch 14, a third vessel branch 16, and a fourth vessel branch 18. Lesions 20 located at a junction 22 reduce blood flow, possibly leading to cardiac arrest. Lesions 20 located at junction 22 are difficult to stent using conventional, tubular bifurcated stents. Although a trifurcated vessel 10 is shown in FIG. 1, those of skill in the art would recognize that the vessel could be a bifurcated vessel, or could include more than four vessels, depending on the area in the body.

FIG. 2 shows an embodiment of a stent 100 in accordance with an embodiment of the present invention. Stent 100 is shown in its expanded form. As shown in FIG. 2, stent 100 is generally spheroidal or ellipsoidal in shape. Such a shape is particularly useful for treating lesions 20 at junction 22 of a trifurcated vessel, for example. Stent 100 comprises longitudinal struts 102 and vertical struts 104. Struts 102, 104 may be made of conventional materials for making stents, such as stainless steel, nickel-chromium alloys, nickel-titanium alloys such as nitinol, polymers, etc. In a preferred embodiment, struts 102, 104 are made of shape memory material, such as a nickel-titanium, such that stent 100 may be a self-expanding stent. Self-expanding stents are placed in a vessel by inserting the stent in a compressed state into the affected region, e.g., an area of stenosis. Once the compressive force is removed, the stent expands to fill the lumen of the vessel. The stent may be compressed using a tube that has a smaller outside diameter than the inner diameter of the affected vessel region. When the stent is released from confinement in the tube, the stent expands to resume its original shape and becomes securely fixed inside the vessel against the vessel wall.

FIG. 3 shows a stent 200 in accordance with another embodiment of the present invention. Stent 200 is shown in its expanded form and is generally spherical in shape. Stent 200 comprises longitudinal struts 202 and vertical struts 204.

As discussed above, stent 100 is generally ellipsoidal or spheroidal in shape. In an ellipsoid or spheroid, any plane section is an ellipse or a circle. Similarly, in a sphere as shown in FIG. 3, any plane section is a circle. Although FIGS. 2 and 3 have been described as ellipsoidal and spherical, respectively, one skilled in the art would recognize that the stents need not be perfect ellipsoids or spheres.

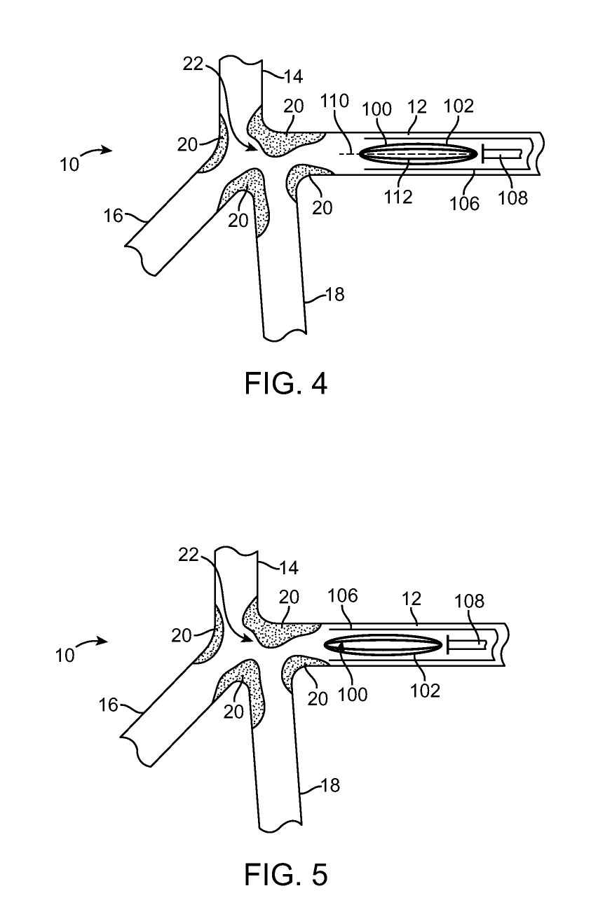

FIGS. 4-7 illustrate schematically a method for delivering stent 100 to junction 22 where four vessels 12, 14, 16, and 18 meet. Stent 100 is delivered through first branch vessel 12 in a compressed state disposed within a sleeve 106, as illustrated in FIG. 4. In its compressed state, stent 100 includes a longitudinal axis 110 that is longer than a transverse axis 112. A pusher 108 is disposed proximal to stent 100 within sleeve 106. Alternatively, sleeve 106 may be disposed around only stent 100 and a catheter may be disposed around both sleeve 106 and pusher 108 for delivery to junction 22.

FIG. 5 illustrates stent 100 delivered adjacent to junction 22. Upon delivery to a position adjacent junction 22, pusher 108 pushes against stent 100 such that stent 100 can move distally without sleeve 106 moving distally. Thus, stent 100 moves distally with respect to sleeve 106. Although a pusher is shown, one skilled in the art of stents would recognize that there are several methods for free a stent from a sleeve, any one of which can be used in conjunction with the present invention.

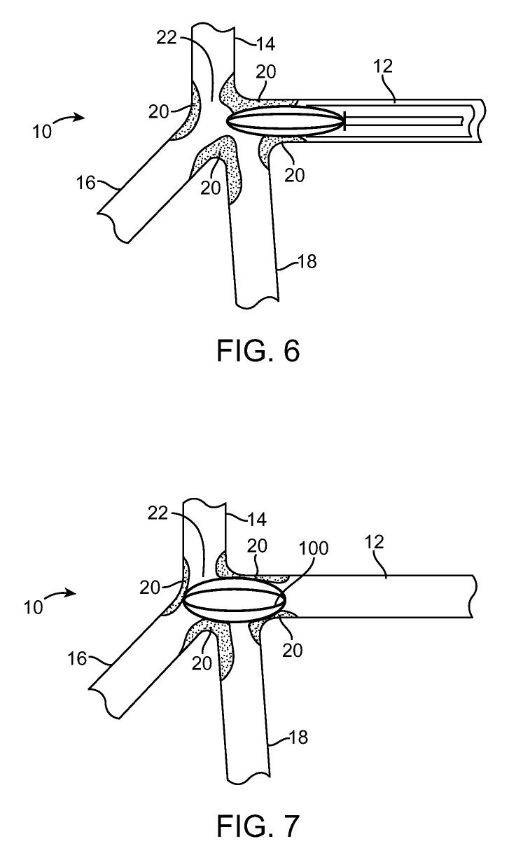

As stent 100 moves distally with respect to sleeve 106, stent 100 begins to expand to its expanded configuration. As noted above, stent 100 is a self-expanding stent. FIG. 6 illustrates stent 100 as it exits sleeve 106, with a portion of stent 100 expanding outside sleeve 106, and a portion of stent 100 still constrained within sleeve 106. As stent 100 expands, it compresses lesions 20 against the vessel walls.

FIG. 7 illustrates stent 100 when stent 100 is completely removed from sleeve 106 and disposed at junction 22. As illustrated in FIG. 7, stent 100 is an ellipsoidal shape and effectively maintains flow through junction 22 and into the branch vessels 12, 14,16, and 18.

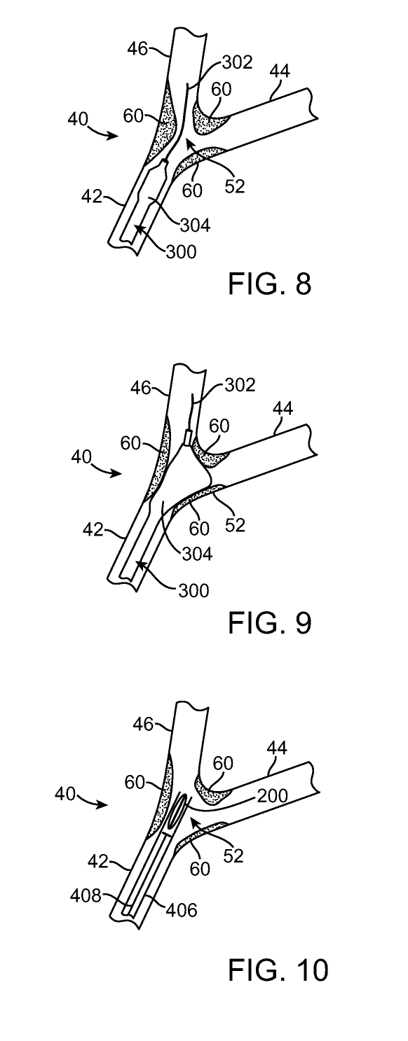

FIGS. 8-12 illustrate schematically a method for delivering stent 200 to junction 52 at bifurcation 40 where a first branch vessel 42, a second branch vessel 44, and a third branch vessel 46 meet. In some cases, stent 200 may not be able to compress lesions 60 against the vessel wall. Accordingly, an angioplasty procedure may be performed prior to delivering stent 200 to the site. In particular, a balloon catheter 300 is delivered to junction 52 along a guidewire 302, as shown in FIG. 8. When a balloon 304 of balloon catheter 300 is disposed at junction 52, balloon 304 is expanded by fluid delivered through catheter 300. Balloon 304 expands to compress lesions 60 against the vessel walls, as illustrated in FIG. 9. The inflation fluid is then drained from balloon 304, balloon 304 returns to its unexpanded state, and catheter 300 is removed.

Stent 200 is delivered then through first branch vessel 42 in a compressed state disposed within a sleeve 406, as illustrated in FIG. 10. A pusher 408 is disposed proximal to stent 200 within sleeve 406. Alternatively, sleeve 406 may be disposed around only stent 200 and a catheter may be disposed around both sleeve 406 and pusher 408 for delivery to junction 52. As illustrated in FIG. 10, sleeve 406 is delivered all the way into junction 52.

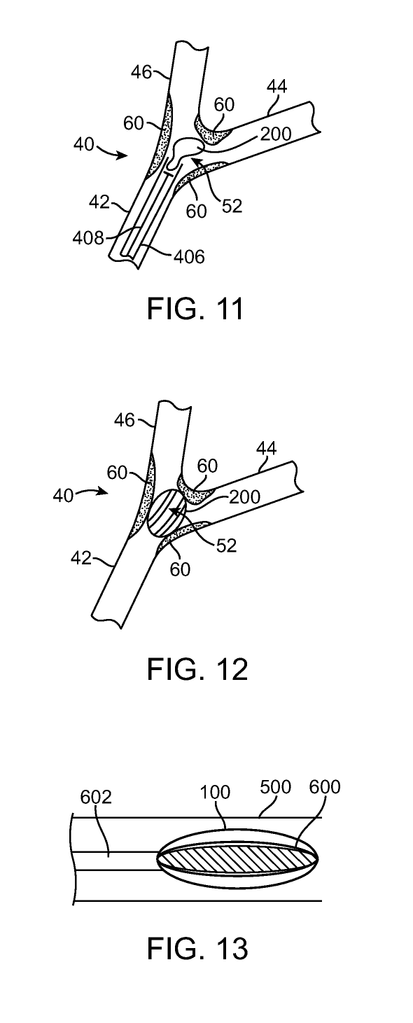

Upon delivery into junction 52, sleeve 406 is retracted while pusher 408 maintains its position, as shown in FIG. 11. Thus, sleeve 406 moves proximally relative to pusher 408, and consequently sleeve 406 moves proximally relative to stent 200. As stent 200 is exposed distal to sleeve 406, stent 200 begins to expand. Upon withdrawal of sleeve 406, stent 200 is completely expanded and remains in place at junction 52, as illustrated in FIG. 12. Stent 200 is a spherical shape and effectively maintains flow through junction 52 and into the branch vessels 42, 44, and 46.

As would be understood by one of ordinary skill in the art, the delivery method described with respect to FIGS. 8-12 can be used with stent 100. Similarly, the delivery described with respect to FIGS. 4-7 can be used for stent 200.

FIG. 13 illustrates stent 100 disposed within a sleeve 500. In FIG. 13, stent 100 is mounted on a balloon 600 disposed at a distal portion of a catheter 602. The delivery system of FIG. 13 operates similar to the previous embodiments described above. However, after stent 100 is disposed at a junction in a vessel, balloon 600 is inflated such that stent 100 is expanded to appose the vessel wall.

While various embodiments of the present invention have been described above, it should be understood that they have been presented by way of illustration and example only, and not limitation. It will be apparent to persons skilled in the relevant art that various changes in form and detail can be made therein without departing from the spirit and scope of the invention. Thus, the breadth and scope of the present invention should not be limited by any of the above-described exemplary embodiments, but should be defined only in accordance with the appended claims and their equivalents. It will also be understood that each feature of each embodiment discussed herein, and of each reference cited herein, can be used in combination with the features of any other embodiment. All patents and publications discussed herein are incorporated by reference herein in their entirety.

What is claimed is:

1. A stent for treating a region of a body lumen wherein at least two vessels form a junction, the stent comprising:
a compressed state wherein the stent includes a longitudinal axis and a transverse axis, wherein a length of the stent along the longitudinal axis is larger than a width of the stent along the transverse axis; and
an expanded state wherein the stent forms a generally ellipsoidal, spheroidal, or spherical shape.

2. The stent of claim 1, wherein the stent is a self-expanding stent.

3. The stent of claim 2, wherein the stent includes a plurality of generally longitudinal struts, wherein said longitudinal struts are made from a shape memory material.

4. The stent of claim 3, wherein said shape memory material is a nickel-titanium alloy.

5. A method for treating a region of a body lumen wherein at least two vessels form a junction, the method comprising the steps of:
disposing a stent within a sleeve, wherein the stent is in a compressed state including a longitudinal axis and a transverse axis, wherein a length of the stent along the longitudinal axis is larger than a width of the stent along the transverse axis;
delivering the sleeve and the stent to the junction; and
withdrawing the sleeve proximally relative to the stent such that the stent is released from the sleeve, wherein the stent expands to form a generally ellipsoidal, spheroidal, or spherical shape.

6. The method of claim 5, further comprising the steps of:
prior to delivering the stent to the junction, delivering a balloon catheter to the junction and expanding the balloon at the junction.

7. The method of claim 5, wherein the stent includes a plurality of generally longitudinal struts, wherein said longitudinal struts are made from a shape memory material.

8. The method of claim 5, further comprising a stopper disposed proximally of the stent such that during the step of withdrawing the sleeve proximally, the stopper prevents the stent from moving proximally such that there is relative movement between the stent and the sleeve.

9. A method for treating a region of a body lumen wherein at least two vessels form a junction, the method comprising the steps of:
disposing a stent within a sleeve and mounted on a balloon catheter, wherein the stent is in a compressed state including a longitudinal axis and a transverse axis, wherein a length of the stent along the longitudinal axis is larger than a width of the stent along the transverse axis;
delivering the sleeve, the balloon catheter, and the stent to the junction;
withdrawing the sleeve proximally relative to the stent such that the stent is released from the sleeve, wherein the stent expands to form a generally ellipsoidal, spheroidal, or spherical shape; and
inflating the balloon to further expand the stent against walls of the body lumen.

10. The method of claim 9, wherein the stent includes a plurality of generally longitudinal struts, wherein said longitudinal struts are made from a shape memory material.
